(19) United States
(12) Patent Application Publication
(10) Pub. No.: US 2026/0306571 A1
(43) Pub. Date: Oct. 8, 2026

(54) CANNABINOID-BASED FORMULATION

(71) Applicant: MRX Medical Limited, Weybridge (GB)

(72) Inventors: Charles MORGAN, London (GB); Melissa STURGESS, London (GB); Nicholas CLARKSON, London (GB)

(21) Appl. No.: 19/483,089

(22) PCT Filed: May 10, 2024
(86) PCT No.: PCT/GB2024/051220

§ 371 Date: Nov. 10, 2025

(30) Foreign Application Priority Data
May 11, 2023 (GB) 2307003.0

(51) Int. Cl. A61K 36/185; A61K 31/00; A61K 47/14

(52) U.S. Cl. CPC A61K 36/3482; A61K 31/658; A61K 47/14; A61K 2236/19; A61K 2236/39; A61K 2236/55

(57) ABSTRACT

The invention relates to cannabinoid-based formulations, and particularly, although not exclusively, to methods for preparing cannabinoid-based formulations, and to the formulations per se, and their use in therapy. The invention is especially concerned with cannabidiol (CBD)-based formulations, their manufacture and uses, for treating various conditions.

The present invention relates to cannabinoid-based formulations, and particularly, although not exclusively, to methods for preparing cannabinoid-based formulations, and to the formulations per se, and their use in therapy. The invention is especially concerned with cannabidiol (CBD)-based formulations, their manufacture and uses, for treating various conditions.

The cannabinoids are several structural classes of compounds found primarily in the cannabis plant. The best studied cannabinoids include cannabidiol (CBD), tetrahydrocannabinal (THC) and cannabinol (CBN), with CBD being the most widely investigated phytocannabinoid (i.e. a plant-derived cannabinoid) due to its therapeutic activities and lack of psychoactive effects. In addition, this compound has shown a large variety of pharmacological effects, such as anti-inflammatory, analgesic and anxiolytic properties and has been used alone for multiple health conditions for many years.

In addition to the pharmaceutical activity of cannabinoids, such as CBD, recent evidence has suggested that terpenes (responsible for the aromas and flavours of many plants) which are also found in the Cannabis sativa plant and other plants in varying amounts and types are also pharmacologically active. Terpenes can, in some instances, bind to the same receptors as cannabinoids, as well as having distinct beneficial actions themselves, and can produce an entourage effect by modulating cannabinoids to result in improved outcomes over either chemical alone. A possible mechanism of the synergistic activity between cannabinoids and terpenes is by promoting the activated form of the cannabinoid receptors, modulation of endocannabinoid synthesis and degradation, and subsequent receptor activation by these endocannabinoids. Additionally, terpenes have been proven to enhance drug permeation via various mechanisms explained in detail elsewhere.

Unfortunately, the CBD that is used for the treatment of various health conditions is usually extracted directly from the cannabis plant, and it is very difficult, if not impossible, to standardise CBD extracts due to a wide variety of factors influencing the biochemical composition of the cannabis plant, such as the geographical location of where the cannabis plant is grown, the fluctuating growth conditions of the plant (i.e. soil and weather conditions), the time of year of CBD extraction from the plant, and differences in extraction methods from the plant, etc. This significant variability in the cannabis plant and extraction parameters results in the CBD formulations that are currently available suffering from the problem of not being consistently reproducible and so not always having the correct amounts of CBD that is listed on the label (Liebling et al., 2022, An Analysis of Over-the-Counter Cannabidiol Products in the United Kingdom, Cannabis and Cannabinoid Research, 7, 2, 207-213).

Furthermore, cannabis plants can have widely different levels and composition of terpenes, which, as mentioned above, are believed to confer a wide range of beneficial activities due to the entourage effect they create by modulating the cannabinoids. However, the extraction methods that are currently used also do not allow the terpenes to be extracted from the cannabis plant in a reproducibly consistent way, and so, as with the CBD, this lack of reproducibility and variability of terpenes between batches means that customers cannot be certain that they are taking exactly the same CBD/terpene mix to treat their condition. This variability in the composition of the CBD formulation from batch to batch results in differences in the therapeutic activity between the batches of the formulation. Moreover, there have been examples where moving from one back to another has had very detrimental effects on patients' health (Booth and Bohlmann, 2019, Terpenes in Cannabis sativa—From plant genome to humans, 284, July, 67-72).

Another problem with currently available CBD formulations is that the direct extraction of CBD from the natural cannabis plant frequently results in differing levels of tetrahydrocannabinol (THC) contamination. THC is a controlled drug and has unwanted addictive and psychotropic activities that are not required for the beneficial activities of CBD for treatment of the various conditions listed above. There are, therefore, strict legal limits on how much THC can be present in a CBD formulation. However, there are safety issues with THC, and current extraction methods mean it cannot be removed completely when the CBD and terpenes are extracted from the cannabis plant, meaning that CBD products are often contaminated with THC (Liebling et al., 2022, An Analysis of Over-the-Counter Cannabidiol Products in the United Kingdom, Cannabis and Cannabinoid Research, 7, 2, 207-213).

Formulations comprising cannabinoids and terpenes are referred to as a Cannabis-Based Product for Medicinal use (CBPM). CBPMs have been added to the treatment paradigm for many patients suffering from acute and chronic inflammatory conditions, pain, and symptoms of negative affect, such as anxiety and depression that have limited or a lack of response to traditional therapeutic approaches. Due to this, the National Institute for Health and Care Excellence (NICE) and the International Association for the Study of Pain (IASP) have made specific calls for research using CBPMs in humans. However, there is still an unmet need for treating multiple treatment-resistant conditions, such as fibromyalgia, chemotherapy-induced peripheral neuropathy (CIPN) and endometriosis which are characterised by pain, inflammation and other negative effects that have an adverse influence on the quality of life of patients suffering from these conditions. Since no one-size-fits-all therapy is currently available for the above mentioned conditions, most patients require multiple and alternative treatment approaches that are often inconvenient and cause multiple side effects. Additionally, whilst numerous CBD formulations are now available as food supplements, making many beneficial health claims, there is minimal assessment of whether they are being administered in a suitable formulation even to achieve blood levels to support these claims, and a recent study analysing a range of CBD containing products in the UK found that there is a great variation in the levels of CBD actually present in these products with varying levels of THC contamination (Liebling et al., 2022, An Analysis of Over-the-Counter Cannabidiol Products in the United Kingdom, Cannabis and Cannabinoid Research, 7, 2, 207-213). However, to date, there are no CBD-containing products available that comply with GMP standards.

Accordingly, there is a need for a new medical CBPM formulation which is produced under defined pharmaceutical standards and under the guidelines of GMP for use in medical research and practice, and in foodstuffs, which therefore facilitates the access and delivery of a pharmaceutical standard CBPM to clinical trials and to benefit patients. There is also a need to produce standardised cannabinoid (e.g. CBD)-based formulations containing reproducibly consistent levels of the desired cannabinoid(s) as well as the associated terpenes while lacking any significant levels of THC. This standardisation means that the composition and biological activity of the CBD formulation will be the same between batches, giving patients significantly more confidence in the CBPM product and its therapeutic effects.

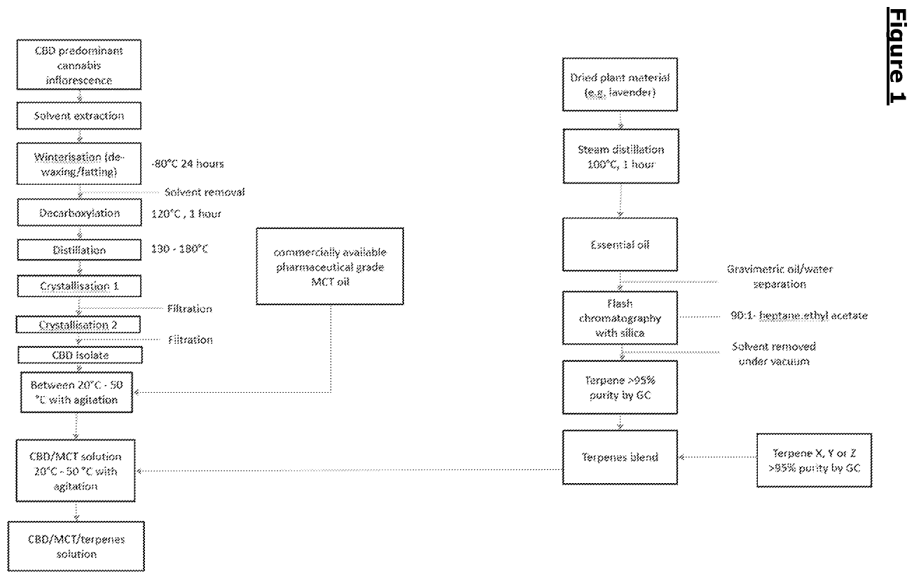

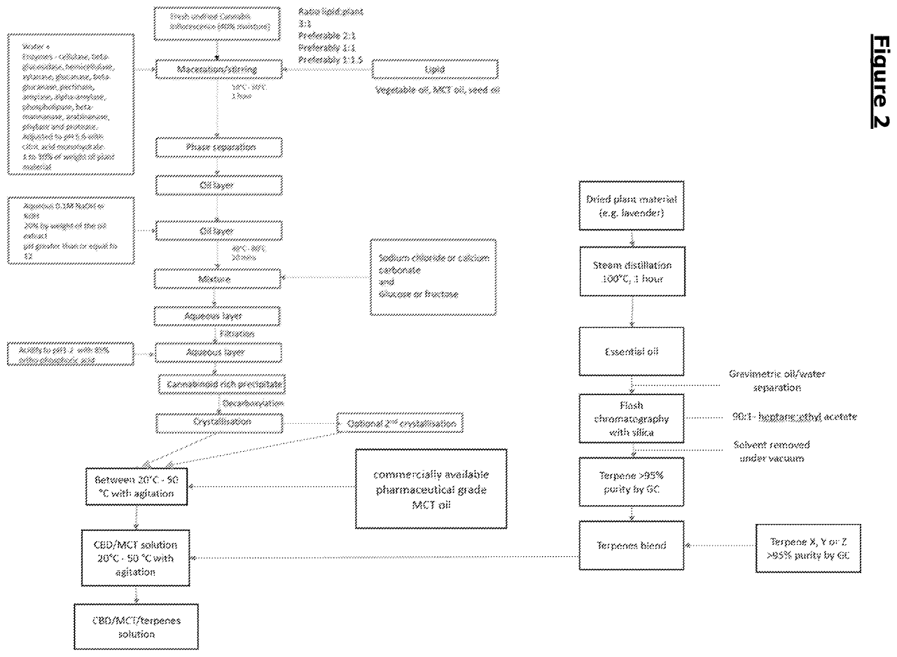

As described in the Examples, and as shown in FIG. 1, the inventors have devised an innovative method for producing a highly standardised formulation comprising the cannabinoid, CBD, and a highly specific mixture of terpenes from scratch using an isolate of CBD of known purity and a highly defined mix of terpenes allowing the preparation of a formulation with standardised amounts of each component, whilst simultaneously minimising any contamination with THC.

Thus, according to a first aspect of the invention, there is provided a method of producing a cannabinoid-based formulation comprising a cannabinoid and substantially lacking tetrahydrocannabinol (THC), the method comprising combining: (i) one or more cannabinoid; and (ii) a plurality of terpenes, to produce a cannabinoid-based formulation, wherein the formulation substantially lacks THC.

In a second aspect, there is provided a cannabinoid-based formulation obtained or obtainable by the method of the first aspect.

In a third aspect of the invention, there is provided the cannabinoid-based formulation of the second aspect, for use in therapy.

In a fourth aspect, there is provided a method of treating, preventing or ameliorating a subject suffering from disease, the method comprising administering, or having administered, to a subject in need of such treatment, a therapeutically active amount of the cannabinoid-based formulation of the second aspect.

Advantageously, and preferably, the method of the first aspect combines the one or more cannabinoid of a known purity, and the plurality of terpenes forming a mixture, also of known purity, to produce the formulation of the second aspect which is fully standardized with a known concentration and purity of each of the constituents, and is fully reproducible, with no variability between different batches. Accordingly, a certificate of analysis can be given for each batch of the formulation of the invention using the method of the first aspect, showing its stability data, as well as evidence that there is substantially no contamination with any THC, thereby providing the patient with full confidence of the composition of the formulation and, therefore, its therapeutic efficacy.

THC may be detected using any standard analytical chemistry approaches, such as liquid chromatography with ultraviolet (UV) or mass spectrometric (MS or MS/MS) detection, e.g. Lukas Vaclavik et al., (2019) ‘Quantitation of Cannabinoids in Cannabis Dried Plant Materials, Concentrates, and Oils Using Liquid Chromatography-Diode Array Detection Technique with Optional Mass Spectrometric Detection: Single-Laboratory Validation Study, First Action 2018.11’ Journal of AOAC INTERNATIONAL, Volume 102, Issue 6, 1 Nov. 2019, Pages 1822-1833.

The concentration of THC in the formulation is preferably less than 1000 μg per finished product, e.g. for 30 ml this equates to 0.0033% (w/v). Therefore, preferably the concentration of THC in the formulation is less than 0.01% (w/v), more preferably less than 0.005% (w/v), and even more preferably less than 0.001% (w/v). Preferably, the concentration of THC in the formulation is less 0.0005% (w/v), and even more preferably less than 0.0002% (w/v), and most preferably less than 0.0001% (w/v). Preferably, however, the formulation comprises no detectable THC using reasonable validated means of analysis.

The term ‘cannabinoid’ can refer to any lipophilic ligand capable of interacting with receptors involved in the endocannabinoid system (ECS). The cannabinoid may comprise a phytocannabinoid (i.e. plant cannabinoid), an endocannabinoid (i.e. endogenous cannabinoids) or a synthetic cannabinoid (i.e. man-made). Traditionally, cannabinoids were thought to bind selectively to the cannabinoid receptors (CB) 1 and 2. However, further research into ECS pharmacology has led to the discovery of various molecular targets for cannabinoids independent of the CBRs 1 and 2, such as the transient receptor potential (TRP) channels and two orphan GPCRs G-protein-coupled receptors (GPR55/18). Chemically, cannabinoids are meroterpenoids (C21-22 terpenophenolic compounds) derived from the alkylation of an alkyl resorcinol with a monoterpene unit. They are mainly synthesised and stored in glandular trichomes, most abundant in female inflorescences of the cannabis plant.

The one or more cannabinoid may be selected from a group of cannabinoids consisting of: cannabidiol (CBD); cannabichromene (CBC); cannabigerol (CBG); and cannabigerol monomethyl ether (CBGM). The one or more cannabinoid may comprise two or more cannabinoids selected from a group consisting of: cannabidiol (CBD); cannabichromene (CBC); cannabigerol (CBG); and cannabigerol monomethyl ether (CBGM). The one or more cannabinoid may comprise three or more cannabinoids selected from a group consisting of: cannabidiol (CBD); cannabichromene (CBC); cannabigerol (CBG); and cannabigerol monomethyl ether (CBGM). The one or more cannabinoid may comprise four or more cannabinoids selected from a group consisting of: cannabidiol (CBD); cannabichromene (CBC); cannabigerol (CBG); and cannabigerol monomethyl ether (CBGM).

Preferably, however, the one or more cannabinoid comprises CBD. The one or more cannabinoid may comprise an isolate of CBD. Preferably, the formulation does not comprise CBC. Preferably, the formulation does not comprise CBG. Preferably, the formulation does not comprise CBGM.

Preferably, the chemical structure of the phytocannabinoid, CBD, used in the method of the invention, comprises Formula [I]:

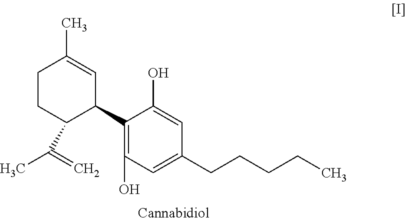 [SMILES: C=C(C)[C@@H]1CCC(C)=C[C@H]1c1c(O)cc(CCCCC)cc1O]

With reference to FIG. 1, in one embodiment, the one or more cannabinoid may be prepared with the use of a solvent (e.g. alcohol or alkane) extraction from plant material (e.g. inflorescence and/or leaves of Cannabis sativa), producing a slurry which may then be filtered to produce a crude liquid extract. The crude extract may then be chilled (to <−20° C. or <−40° C. for at least 12 hours, and preferably−80° C. for 24 hours) to precipitate fats and waxes that are subsequently filtered off to produce a ‘winterised’ extract. The solvent may then be removed under reduced pressure and the remaining crude oil is then preferably ‘decarboxylated’ to convert the acidic compounds to neutral forms, which is typically performed at >100° C. for at least 20 minutes, more preferably 120° C. for at least 30 minutes. The resulting decarboxylated extract may then be further purified by distillation to remove residual extraneous non-cannabinoid materials. The distillation step may be carried out at between 10° and 200° C., preferably between 130-180° C., and may be short path distillation. The resulting material may be dissolved in a solvent (e.g. alcohol) to around the saturation point of the solution, before an anti-solvent (e.g. alkane) is (slowly) added to induce crystallisation. Upon completion of the first crystallisation (~95% purity cannabinoid, preferably CBD), the solid material may then be filtered off and re-dissolved in alcohol, and a second crystallisation may be performed to produce an isolated cannabinoid (preferably, CBD) of the required specification.

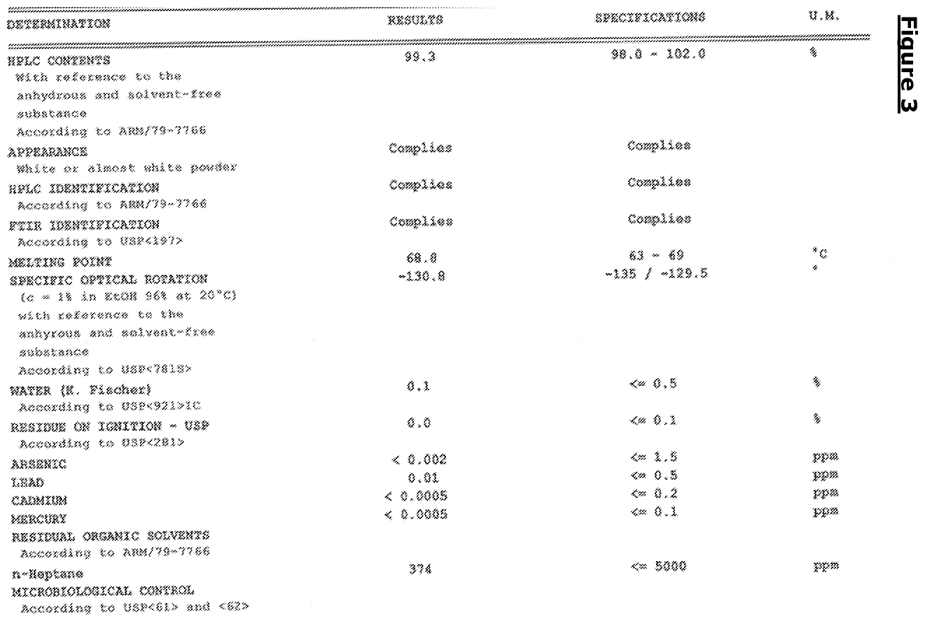

With reference to FIG. 2, in a preferred embodiment, the one or more cannabinoid may be prepared by using enzyme digestion of plant material (e.g. inflorescence and/or leaves of Cannabis sativa). The plant material may comprise a moisture content of at least 20%, preferably at least 30%, more preferably at least 40%. Preferably, the plant material is contacted with an enzyme solution. The enzyme solution preferably comprises one or more enzyme selected from a group of enzymes consisting of: cellulase, beta-glucosidase, hemicellulase, xylanase, glucanase, beta-glucanase, pectinase, amylase, alpha-amylase, phospholipase, beta-mannanase, arabinanase, phytase and protease. Preferably, the pH of the enzyme solution is adjusted to at least about pH4, more preferably at least about pH5, and even more preferably about pH5.6. The pH adjustment may be achieved using citric acid. A weight ratio of 1:20 of aqueous enzyme solution relative to plant material may be used. Preferably, about 1-10% (wt) pf plant material is used to 99-10% (wt) enzyme solution. The plant material is preferably macerated before, during and/or after contacting with the enzyme solution.

Preferably, the plant material is contacted with a lipid for extracting plant compounds of interest (i.e. the cannabinoid). For example, the lipid may be vegetable oil, MCT oil, seed oil or olive oil. The weight ratio of lipid to plant material is about 2:1, preferably 1:1, more preferably 1:1.5. The plant material is preferably macerated before, during and/or after contacting with the lipid. The mixture is preferably stirred and macerated. This mixture is preferably agitated for at least 30, 40 or 60 minutes at a temperature of between about 5° C. and 80° C., more preferably between 8° C. and 65° C., and most preferably between 10° C. and 50° C. Upon full breakdown and dissolution of the plant material, the oil and water phases are preferably separated by gravity, centrifugation, filtration or a combination thereof. The oil layer is preferably transferred to a separate vessel having agitation capability. The oil layer is preferably contacted with an aqueous base. The base may be an aqueous solution of 0.1M NaOH or KOH, preferably at a pH greater than or equal to 12. The weight of aqueous basic solution may be 1:3 ratio by weight relative to the cannabinoid-containing oil, preferably 1:4, more preferably 1:5. This mixture is preferably agitated for at least 10-20 minutes at a temperature of between about 20° C. and 100° C., more preferably between 30° C. and 90° C., and most preferably between 40° C. and 80° C. On completion of the previous step, the mixture is preferably contacted with a combination of: (i) sodium chloride and/or calcium carbonate; and (ii) glucose and/or fructose. Preferably, this mixture is agitated for a further 10, 15 or 20 minutes.

Upon completion, the layers are preferably separated by gravity or centrifugation, and the aqueous layer may then be separated off. Preferably, the pH of the separated aqueous layer is reduced to a pH of less than 3, 2 or 1. This may be achieved by contacting the aqueous layer with acid, more preferably phosphoric acid (e.g. 85% ortho-phosphoric acid). Preferably, the solution is agitated for a further 10, 15 or 20 minutes, causing precipitation of cannabinoid acid salts. Preferably, this liquid is filtered, giving a cannabinoid-rich solid precipitate between 65%-90% total cannabinoid content. The cannabinoid-rich precipitate may then be heated to at least 90, 100 or 120° C. for at least 30 mins, 45 mins or 60 mins, or until such time that complete decarboxylation has been achieved. The resulting material may be dissolved in a solvent (e.g. alcohol) to around the saturation point of the solution, before an anti-solvent (e.g. alkane) is (slowly) added to induce crystallisation. Optionally, the solid material may then be filtered off and re-dissolved in solvent (e.g. alcohol), and a second crystallisation may be performed to produce an isolated cannabinoid (preferably, CBD) of the required specification.

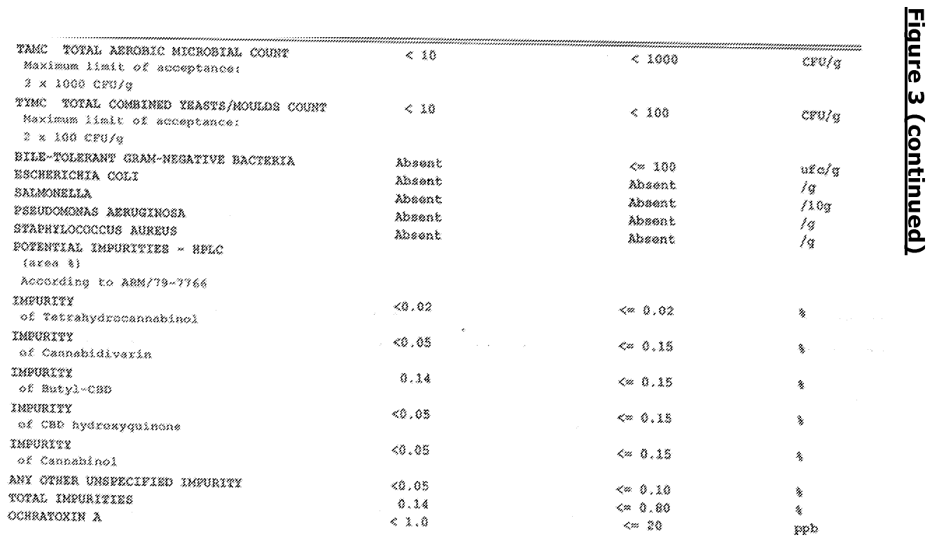

The purity of the one or more cannabinoid (preferably, CBD) may be at least 97%, at least 98% or at least 99%, or about 98-100%, as measured by HPLC-UV assay at 220 nm detection wavelength. As shown in FIG. 3, the inventors have a certificate of analysis (COA) for the CBD isolate. The specification has been set in order to comply with ICH Q3A guidance on impurities in new Drug Substance, i.e. to demonstrate sufficient control of Organic impurities (minor cannabinoids, solvents etc.), inorganic impurities (e.g. heavy metals) and microbiological contamination. Particularly relevant is the decision to set the specification for d9-tetrahydrocannabinol to 0.02%, which is lower than the requirements of less than, or equal to, 0.15% for any single known organic impurity.

The concentration of the one or more cannabinoid (preferably, CBD) in the cannabinoid-based formulation may be at least 2% (w/v), at least 3% (w/v) or at least 4% (w/v). The concentration of the one or more cannabinoid (preferably, CBD) in the cannabinoid-based formulation may be at least 5% (w/v), at least 6% (w/v) or at least 7% (w/v). Preferably, the concentration of the one or more cannabinoid (preferably, CBD) in the cannabinoid-based formulation may be at least 8% (w/v), at least 9% (w/v) or at least 10% (w/v). Preferably, the concentration of the one or more cannabinoid (preferably, CBD) in the cannabinoid-based formulation may be at least 11% (w/v), or at least 12% (w/v).

The concentration of the one or more cannabinoid (preferably, CBD) in the cannabinoid-based formulation may be less than 20% (w/v), less than 19% (w/v) or less than 18% (w/v). The concentration of the one or more cannabinoid (preferably, CBD) in the cannabinoid-based formulation may be less than 17% (w/v), less than 16% (w/v) or less than 15% (w/v). Preferably, the concentration of the one or more cannabinoid (preferably, CBD) in the cannabinoid-based formulation may be less than 14% (w/v), less than 13% (w/v) or less than 12% (w/v). Preferably, the concentration of the one or more cannabinoid (preferably, CBD) in the cannabinoid-based formulation may be less than 11% (w/v), less than 10% (w/v) or less than 9% (w/v).

The concentration of the one or more cannabinoid (preferably, CBD) in the cannabinoid-based formulation may be between 1 and 25% (w/v), between 3 and 20% (w/v) or between 5 and 17% (w/v). Preferably, the concentration of the one or more cannabinoid (preferably, CBD) in the cannabinoid-based formulation is between 7 and 13% (w/v), between 8 and 12% (w/v) or between 9 and 11% (w/v). Preferably, the concentration of the one or more cannabinoid (preferably, CBD) in the cannabinoid-based formulation is about 10% (w/v). It will be appreciated that any of the lower values for the concentration of the one or more cannabinoid mentioned herein may be combined with any of the upper values for the concentration of the one or more cannabinoid.

Terpenes are lipids (complex compounds of fatty acids) and belong to the terpenoid family. Chemically, terpenes present a distinctive carbon skeleton which consists of a basic five-carbon isoprene unit (C5H8, 2-methyl-1,3-butadiene) linked together most commonly in a head-to-tail arrangement. However, they can be built in other configurations with varying degrees of oxidation, unsaturation, functional groups and ring closures, leading to a vast diversity of structural classes. Nevertheless, terpenes can be grouped into categories according to the number of isoprene units (n) in the molecule. Isoprene is a gaseous hydrocarbon released by multiple plants as a natural by-product of plant metabolism. However, some of the larger and more complex terpenes, such as squalene and lanosterol, are also found in animals.

As illustrated in FIGS. 1 and 2, the plurality of terpenes may be prepared by first steam distillation from the appropriate species of plant to produce an essential oil. The resulting essential oil may then be purified by chromatography (preferably by flash chromatography) to separate the individual terpene components at the 5 required purity.

The purity of the plurality of terpenes may be at least 90%, preferably at least 92% and more preferably at least 95% of total terpenes as measured by GC-MS.

The plurality of terpenes may be selected from a group of terpenes shown in Table 1.

TABLE 1
Classification of Terpenes

| Classification | Isoprene units (n) | Carbon Atoms |
|---|---|---|
| Monoterpenes | 2 | 10 |
| Sesquiterpenes | 3 | 15 |
| Diterpenes | 4 | 20 |
| Sesterpenes | 5 | 25 |
| Triterpenes | 6 | 30 |
| Tetraterpenes | 8 | 40 |
| Polyterpenes | >100 | 50 |

Thus, the plurality of terpenes may be selected from a group of terpenes consisting of: monoterpenes, sesquiterpenes, diterpenes, sesterpenes, triterpenes, tetraterpenes and polyterpenes.

Preferably, the monoterpene is selected from a group of monoterpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Geraniol and Fenchol.

Preferably, the sesquiterpene is selected from a group of sesquiterpenes consisting of: Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, and Bisbolene.

The chemical structure of the terpenes which may be present in the formulation are shown below. Terpenes are grouped depending on the carbon number.

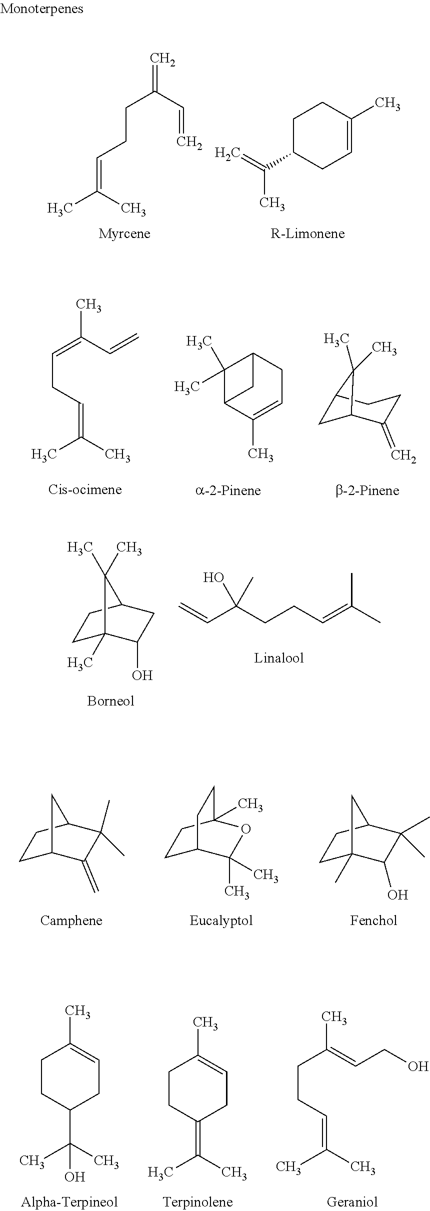

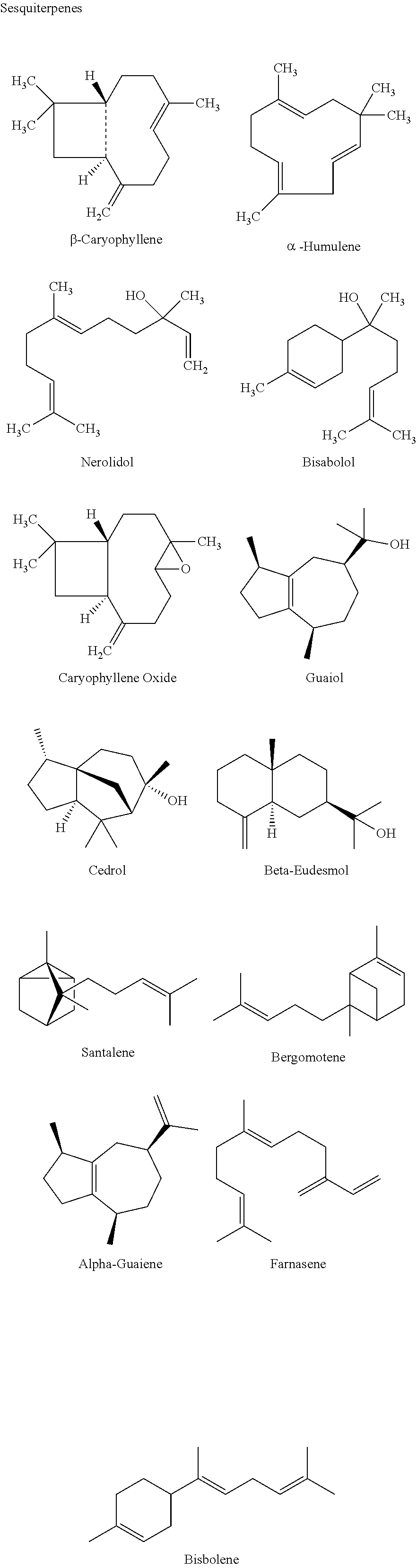

Hence, the plurality of terpenes may be selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.

Preferably, the plurality of terpenes comprises at least three terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.

Preferably, the plurality of terpenes comprises at least four terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.

Preferably, the plurality of terpenes comprises at least five terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.

Preferably, the plurality of terpenes comprises at least six terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.

Preferably, the plurality of terpenes comprises at least seven terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.

Preferably, the plurality of terpenes comprises at least eight terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.

Preferably, the plurality of terpenes comprises at least nine terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.

Preferably, the plurality of terpenes comprises at least ten terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.

Preferably, the plurality of terpenes comprises at least eleven terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.

Preferably, the plurality of terpenes comprises at least twelve terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.

Preferably, the plurality of terpenes comprises at least thirteen terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.

Preferably, the plurality of terpenes comprises at least fourteen terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.

Preferably, the plurality of terpenes comprises at least fifteen terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.

As described in the Examples, the inventors have synthesised a preferred embodiment of the cannabinoid-based formulation of the invention with a specific composition of terpenes. It also includes a cannabinoid (i.e. CBD), and an excipient or carrier (e.g. a MCT oil). In one embodiment, the composition is the formulation as summarised in Table 3, and its terpene mix is described below.

Preferably, the plurality of terpenes comprises at least two, three, four or five terpenes selected from a group of terpenes consisting of: Alpha-Pinene, Beta-Pinene, Limonene, Cis-Ocimene, Beta-Caryophyllene, Alpha-Humulene, Beta-Myrcene, Terpinolene, Trans Beta Farnasene, Linalool, Bisabolol, Alpha-Phellandrene, Gamma-Terpinene, Geraniol and Alpha-Terpineol.

Preferably, the plurality of terpenes comprises at least six, seven or eight terpenes selected from a group of terpenes consisting of: Alpha-Pinene, Beta-Pinene, Limonene, Cis-Ocimene, Beta-Caryophyllene, Alpha-Humulene, Beta-Myrcene, Terpinolene, Trans Beta Farnasene, Linalool, Bisabolol, Alpha-Phellandrene, Gamma-Terpinene, Geraniol and Alpha-Terpineol.

Preferably, the plurality of terpenes comprises at least nine, ten or eleven terpenes selected from a group of terpenes consisting of: Alpha-Pinene, Beta-Pinene, Limonene, Cis-Ocimene, Beta-Caryophyllene, Alpha-Humulene, Beta-Myrcene, Terpinolene, Trans Beta Farnasene, Linalool, Bisabolol, Alpha-Phellandrene, Gamma-Terpinene, Geraniol and Alpha-Terpineol.

Preferably, the plurality of terpenes comprises at least twelve, thirteen or fourteen terpenes selected from a group of terpenes consisting of: Alpha-Pinene, Beta-Pinene, Limonene, Cis-Ocimene, Beta-Caryophyllene, Alpha-Humulene, Beta-Myrcene, Terpinolene, Trans Beta Farnasene, Linalool, Bisabolol, Alpha-Phellandrene, Gamma-Terpinene, Geraniol and Alpha-Terpineol.

Thus, in a preferred embodiment of the cannabinoid-based formulation of the invention, the plurality of terpenes comprises α-Pinene.

The concentration of α-Pinene in the cannabinoid-based formulation may be at least 0.0001 (w/v), or at least 0.001% (w/v). Preferably, the concentration of α-Pinene in the cannabinoid-based formulation may be at least 0.005% (w/v), or at least 0.01% (w/v). The concentration of α-Pinene in the cannabinoid-based formulation may be less than 0.75% (w/v), less than 0.5% (w/v) or less than 0.3% (w/v). The concentration of α-Pinene in the cannabinoid-based formulation may be less than 0.2% (w/v), or less than 0.15% (w/v). The concentration of α-Pinene in the cannabinoid-based formulation may be between 0.0001 and 0.5% (w/v), between 0.01 and 0.3% (w/v) or between 0.05 and 0.2% (w/v). Preferably, the concentration of α-Pinene in the cannabinoid-based formulation is about 0.1% (w/v). It will be appreciated that any of the lower values for the concentration of α-Pinene mentioned herein may be combined with any of the upper values for the concentration of α-Pinene.

In a preferred embodiment, the plurality of terpenes comprises β-Pinene.

The concentration of β-Pinene in the cannabinoid-based formulation may be at least 0.0001 (w/v), or at least 0.001% (w/v). Preferably, the concentration of β-Pinene in the cannabinoid-based formulation may be at least 0.005% (w/v), or at least 0.01% (w/v). The concentration of β-Pinene in the cannabinoid-based formulation may be less than 0.75% (w/v), less than 0.5% (w/v) or less than 0.3% (w/v). The concentration of β-Pinene in the cannabinoid-based formulation may be less than 0.2% (w/v), or less than 0.15% (w/v). The concentration of 3-Pinene in the cannabinoid-based formulation may be between 0.0001 and 0.5% (w/v), between 0.01 and 0.3% (w/v) or between 0.05 and 0.2% (w/v). Preferably, the concentration of β-Pinene in the cannabinoid-based formulation is about 0.1% (w/v). It will be appreciated that any of the lower values for the concentration of β-Pinene mentioned herein may be combined with any of the upper values for the concentration of β-Pinene.

In a preferred embodiment, the plurality of terpenes comprises Limonene.

The concentration of Limonene in the cannabinoid-based formulation may be at least 0.0001 (w/v), or at least 0.001% (w/v). Preferably, the concentration of Limonene in the cannabinoid-based formulation may be at least 0.005% (w/v), or at least 0.01% (w/v). The concentration of Limonene in the cannabinoid-based formulation may be less than 0.75% (w/v), less than 0.5% (w/v) or less than 0.3% (w/v). The concentration of Limonene in the cannabinoid-based formulation may be less than 0.2% (w/v), or less than 0.15% (w/v). The concentration of Limonene in the cannabinoid-based formulation may be between 0.0001 and 0.5% (w/v), between 0.01 and 0.3% (w/v) or between 0.05 and 0.2% (w/v). Preferably, the concentration of Limonene in the cannabinoid-based formulation is about 0.1% (w/v). It will be appreciated that any of the lower values for the concentration of Limonene mentioned herein may be combined with any of the upper values for the concentration of Limonene.

In a preferred embodiment, the plurality of terpenes comprises Cis-Ocimene.

The concentration of Cis-Ocimene in the cannabinoid-based formulation may be at least 0.0001 (w/v), or at least 0.001% (w/v). Preferably, the concentration of Cis-Ocimene in the cannabinoid-based formulation may be at least 0.005% (w/v), or at least 0.01% (w/v). The concentration of Cis-Ocimene in the cannabinoid-based formulation may be less than 0.75% (w/v), less than 0.5% (w/v) or less than 0.3% (w/v). The concentration of Cis-Ocimene in the cannabinoid-based formulation may be less than 0.2% (w/v), or less than 0.15% (w/v). The concentration of Cis-Ocimene in the cannabinoid-based formulation may be between 0.0001 and 0.5% (w/v), between 0.01 and 0.3% (w/v) or between 0.05 and 0.2% (w/v). Preferably, the concentration of Cis-Ocimene in the cannabinoid-based formulation is about 0.1% (w/v). It will be appreciated that any of the lower values for the concentration of Cis-Ocimene mentioned herein may be combined with any of the upper values for the concentration of Cis-Ocimene.

In a preferred embodiment, the plurality of terpenes comprises β-Caryophyllene.

The concentration of β-Caryophyllene in the cannabinoid-based formulation may be at least 0.0001 (w/v), or at least 0.001% (w/v). Preferably, the concentration of β-Caryophyllene in the cannabinoid-based formulation may be at least 0.005% (w/v), or at least 0.01% (w/v). The concentration of β-Caryophyllene in the cannabinoid-based formulation may be less than 0.75% (w/v), less than 0.5% (w/v) or less than 0.3% (w/v). The concentration of β-Caryophyllene in the cannabinoid-based formulation may be less than 0.2% (w/v), or less than 0.15% (w/v). The concentration of β-Caryophyllene in the cannabinoid-based formulation may be between 0.0001 and 0.5% (w/v), between 0.01 and 0.3% (w/v) or between 0.05 and 0.2% (w/v). Preferably, the concentration of β-Caryophyllene in the cannabinoid-based formulation is about 0.1% (w/v). It will be appreciated that any of the lower values for the concentration of β-Caryophyllene mentioned herein may be combined with any of the upper values for the concentration of β-Caryophyllene.

In a preferred embodiment, the plurality of terpenes comprises Alpha-Humulene.

The concentration of Alpha-Humulene in the cannabinoid-based formulation may be at least 0.0001 (w/v), or at least 0.001% (w/v). Preferably, the concentration of Alpha-Humulene in the cannabinoid-based formulation may be at least 0.005% (w/v), or at least 0.01% (w/v). The concentration of Alpha-Humulene in the cannabinoid-based formulation may be less than 0.75% (w/v), less than 0.5% (w/v) or less than 0.3% (w/v). The concentration of Alpha-Humulene in the cannabinoid-based formulation may be less than 0.2% (w/v), or less than 0.15% (w/v). The concentration of Alpha-Humulene in the cannabinoid-based formulation may be between 0.0001 and 0.5% (w/v), between 0.01 and 0.3% (w/v) or between 0.05 and 0.2% (w/v). Preferably, the concentration of Alpha-Humulene in the cannabinoid-based formulation is about 0.1% (w/v). It will be appreciated that any of the lower values for the concentration of Alpha-Humulene mentioned herein may be combined with any of the upper values for the concentration of Alpha-Humulene.

In the first preferred embodiment, the plurality of terpenes comprises Myrcene.

The concentration of Myrcene in the cannabinoid-based formulation may be at least 0.0001 (w/v), or at least 0.001% (w/v). Preferably, the concentration of Myrcene in the cannabinoid-based formulation may be at least 0.005% (w/v), or at least 0.01% (w/v). The concentration of Myrcene in the cannabinoid-based formulation may be less than 0.75% (w/v), less than 0.5% (w/v) or less than 0.3% (w/v). The concentration of Myrcene in the cannabinoid-based formulation may be less than 0.2% (w/v), or less than 0.15% (w/v). The concentration of Myrcene in the cannabinoid-based formulation may be between 0.0001 and 0.5% (w/v), between 0.01 and 0.3% (w/v) or between 0.05 and 0.2% (w/v). Preferably, the concentration of Myrcene in the cannabinoid-based formulation is about 0.1% (w/v). It will be appreciated that any of the lower values for the concentration of Myrcene mentioned herein may be combined with any of the upper values for the concentration of Myrcene.

In a preferred embodiment, the plurality of terpenes comprises Terpinolene.

The concentration of Terpinolene in the cannabinoid-based formulation may be at least 0.0001 (w/v), or at least 0.001% (w/v). Preferably, the concentration of Terpinolene in the cannabinoid-based formulation may be at least 0.005% (w/v), or at least 0.01% (w/v). The concentration of Terpinolene in the cannabinoid-based formulation may be less than 0.75% (w/v), less than 0.5% (w/v) or less than 0.3% (w/v). The concentration of Terpinolene in the cannabinoid-based formulation may be less than 0.2% (w/v), or less than 0.15% (w/v). The concentration of Terpinolene in the cannabinoid-based formulation may be between 0.0001 and 0.5% (w/v), between 0.01 and 0.3% (w/v) or between 0.05 and 0.2% (w/v). Preferably, the concentration of Terpinolene in the cannabinoid-based formulation is about 0.1% (w/v). It will be appreciated that any of the lower values for the concentration of Terpinolene mentioned herein may be combined with any of the upper values for the concentration of Terpinolene.

In a preferred embodiment, the plurality of terpenes comprises Farnesene.

The concentration of Farnesene in the cannabinoid-based formulation may be at least 0.0001 (w/v), or at least 0.001% (w/v). Preferably, the concentration of Farnesene in the cannabinoid-based formulation may be at least 0.005% (w/v), or at least 0.01% (w/v). The concentration of Farnesene in the cannabinoid-based formulation may be less than 0.75% (w/v), less than 0.5% (w/v) or less than 0.3% (w/v). The concentration of Farnesene in the cannabinoid-based formulation may be less than 0.2% (w/v), or less than 0.15% (w/v). The concentration of Farnesene in the cannabinoid-based formulation may be between 0.0001 and 0.5% (w/v), between 0.01 and 0.3% (w/v) or between 0.05 and 0.2% (w/v). Preferably, the concentration of Farnesene in the cannabinoid-based formulation is about 0.1% (w/v). It will be appreciated that any of the lower values for the concentration of Farnesene mentioned herein may be combined with any of the upper values for the concentration of Farnesene.

In the first preferred embodiment, the plurality of terpenes comprises Linalool.

The concentration of Linalool in the cannabinoid-based formulation may be at least 0.0001 (w/v), or at least 0.001% (w/v). Preferably, the concentration of Linalool in the cannabinoid-based formulation may be at least 0.005% (w/v), or at least 0.01% (w/v). The concentration of Linalool in the cannabinoid-based formulation may be less than 0.75% (w/v), less than 0.5% (w/v) or less than 0.3% (w/v). The concentration of Linalool in the cannabinoid-based formulation may be less than 0.2% (w/v), or less than 0.15% (w/v). The concentration of Linalool in the cannabinoid-based formulation may be between 0.0001 and 0.5% (w/v), between 0.01 and 0.3% (w/v) or between 0.05 and 0.2% (w/v). Preferably, the concentration of Linalool in the cannabinoid-based formulation is about 0.1% (w/v). It will be appreciated that any of the lower values for the concentration of Linalool mentioned herein may be combined with any of the upper values for the concentration of Linalool.

In a preferred embodiment, the plurality of terpenes comprises Bisabolol.

The concentration of Bisabolol in the cannabinoid-based formulation may be at least 0.0001 (w/v), or at least 0.001% (w/v). Preferably, the concentration of Bisabolol in the cannabinoid-based formulation may be at least 0.005% (w/v), or at least 0.01% (w/v). The concentration of Bisabolol in the cannabinoid-based formulation may be less than 0.75% (w/v), less than 0.5% (w/v) or less than 0.3% (w/v). The concentration of Bisabolol in the cannabinoid-based formulation may be less than 0.2% (w/v), or less than 0.15% (w/v). The concentration of Bisabolol in the cannabinoid-based formulation may be between 0.0001 and 0.5% (w/v), between 0.01 and 0.3% (w/v) or between 0.05 and 0.2% (w/v). Preferably, the concentration of Bisabolol ene in the cannabinoid-based formulation is about 0.1% (w/v). It will be appreciated that any of the lower values for the concentration of Bisabolol mentioned herein may be combined with any of the upper values for the concentration of Bisabolol.

In a preferred embodiment, the plurality of terpenes comprises Alpha-Phellandrene.

The concentration of Alpha-Phellandrene in the cannabinoid-based formulation may be at least 0.0001 (w/v), or at least 0.001% (w/v). Preferably, the concentration of Alpha-Phellandrene in the cannabinoid-based formulation may be at least 0.005% (w/v), or at least 0.01% (w/v). The concentration of Alpha-Phellandrene in the cannabinoid-based formulation may be less than 0.75% (w/v), less than 0.5% (w/v) or less than 0.3% (w/v). The concentration of Alpha-Phellandrene in the cannabinoid-based formulation may be less than 0.2% (w/v), or less than 0.15% (w/v). The concentration of Alpha-Phellandrene in the cannabinoid-based formulation may be between 0.0001 and 0.5% (w/v), between 0.01 and 0.3% (w/v) or between 0.05 and 0.2% (w/v). Preferably, the concentration of Alpha-Phellandrene in the cannabinoid-based formulation is about 0.1% (w/v). It will be appreciated that any of the lower values for the concentration of Alpha-Phellandrene mentioned herein may be combined with any of the upper values for the concentration of Alpha-Phellandrene.

In a preferred embodiment, the plurality of terpenes comprises Gamma-Terpinene.

The concentration of Gamma-Terpinene in the cannabinoid-based formulation may be at least 0.0001 (w/v), or at least 0.001% (w/v). Preferably, the concentration of Gamma-Terpinene in the cannabinoid-based formulation may be at least 0.005% (w/v), or at least 0.01% (w/v). The concentration of Gamma-Terpinene in the cannabinoid-based formulation may be less than 0.75% (w/v), less than 0.5% (w/v) or less than 0.3% (w/v). The concentration of Gamma-Terpinene in the cannabinoid-based formulation may be less than 0.2% (w/v), or less than 0.15% (w/v). The concentration of Gamma-Terpinene in the cannabinoid-based formulation may be between 0.0001 and 0.5% (w/v), between 0.01 and 0.3% (w/v) or between 0.05 and 0.2% (w/v). Preferably, the concentration of Gamma-Terpinene in the cannabinoid-based formulation is about 0.1% (w/v). It will be appreciated that any of the lower values for the concentration of Gamma-Terpinene mentioned herein may be combined with any of the upper values for the concentration of Gamma-Terpinene.

In a preferred embodiment, the plurality of terpenes comprises Geraniol.

The concentration of Geraniol in the cannabinoid-based formulation may be at least 0.0001 (w/v), or at least 0.001% (w/v). Preferably, the concentration of Geraniol in the cannabinoid-based formulation may be at least 0.005% (w/v), or at least 0.01% (w/v). The concentration of Geraniol in the cannabinoid-based formulation may be less than 0.75% (w/v), less than 0.5% (w/v) or less than 0.3% (w/v). The concentration of Geraniol in the cannabinoid-based formulation may be less than 0.2% (w/v), or less than 0.15% (w/v). The concentration of Geraniol in the cannabinoid-based formulation may be between 0.0001 and 0.5% (w/v), between 0.01 and 0.3% (w/v) or between 0.05 and 0.2% (w/v). Preferably, the concentration of Geraniol in the cannabinoid-based formulation is about 0.1% (w/v). It will be appreciated that any of the lower values for the concentration of Geraniol mentioned herein may be combined with any of the upper values for the concentration of Geraniol.

In a preferred embodiment, the plurality of terpenes comprises Alpha-Terpineol.

The concentration of Alpha-Terpineol in the cannabinoid-based formulation may be at least 0.0001 (w/v), or at least 0.001% (w/v). Preferably, the concentration of Alpha-Terpineol in the cannabinoid-based formulation may be at least 0.005% (w/v), or at least 0.01% (w/v). The concentration of Alpha-Terpineol in the cannabinoid-based formulation may be less than 0.75% (w/v), less than 0.5% (w/v) or less than 0.3% (w/v). The concentration of Alpha-Terpineol in the cannabinoid-based formulation may be less than 0.2% (w/v), or less than 0.15% (w/v). The concentration of Alpha-Terpineol in the cannabinoid-based formulation may be between 0.0001 and 0.5% (w/v), between 0.01 and 0.3% (w/v) or between 0.05 and 0.2% (w/v). Preferably, the concentration of Alpha-Terpineol in the cannabinoid-based formulation is about 0.1% (w/v). It will be appreciated that any of the lower values for the concentration of Alpha-Terpineol mentioned herein may be combined with any of the upper values for the concentration of Alpha-Terpineol.

Preferably, the method comprises combining the cannabinoid and plurality of terpenes with a pharmaceutically acceptable excipient or carrier.

Preferably, the method comprises contacting the cannabinoid with the pharmaceutically acceptable excipient or carrier at a temperature of between about 10° C. and 70° C., preferably between about 15° C. and 60° C., and more preferably between about 20° C. and 50° C., to form a mixture of the cannabinoid and excipient or carrier. Preferably, the mixture of cannabinoid and excipient or carrier is agitated.

Preferably, the mixture of cannabinoid and excipient or carrier is contacted with the plurality of terpenes at a temperature of between about 10° C. and 70° C., preferably between about 15° C. and 60° C., and more preferably between about 20° C. and 50° C., to create the formulation of the invention.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a liquid, and the pharmaceutical composition is in the form of a suspension in solution. Liquid vehicles are used in preparing solutions, suspensions, emulsions, syrups, elixirs and pressurized compositions. The cannabinoid and terpenes may be dissolved or suspended in a pharmaceutically acceptable liquid vehicle such as water, alcohol, ionic buffered solution, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil).

Pharmaceutically acceptable carriers, excipients, and diluents are relatively inert substances that facilitate administration or a pharmaceutically effective substance and can be supplied as a liquid solutions or suspensions, as emulsions, or as solid forms suitable for dissolution or suspension in liquid prior to use. For example, an excipient can give forms suitable for consistency, or act as a diluent. Suitable excipients include, but are not limited to stabilizing agents, wetting and emulsifying agents, salts for varying osmolarity, encapsulating agents, pH buffering substances, and buffers. Such excipients include any pharmaceutical agent suitable for direct delivery to the subject (for example sub-lingually) which may be administered without undue toxicity. Pharmaceutical acceptable excipients include, but are not limited to, sorbitol, any of the various TWEEN compounds, and liquids such as water, saline, glycerol and ethanol. Pharmaceutically acceptable salts can be included therein, for example, mineral acid salts such as hydrochlorides, hydrobromides, phosphates, sulphates and the like; and the salts of organic acids such as acetates, propionates, malonates, or benzoates.

In some embodiments, pharmaceutical acceptable excipients may include pharmaceutical acceptable carriers. Such pharmaceutically acceptable carriers can be sterile liquids, such as water and oil, including those of petroleum, animal, vegetable or synthetic origin, such as peanut oil, soybean oil, or mineral oil. Saline solutions and aqueous dextrose, polyethylene glycol (PEG) and glycerol solutions can also be employed as liquid carriers, particularly for sub-lingual solutions. Additional ingredients may also be used, for example preservatives, buffers, tonicity agents, antioxidants and stabilizers, non-ionic wetting or clarifying agents, or viscosity-increasing agents. A thorough discussion of pharmaceutical acceptable excipients and carriers is available in Remington's Pharmaceutical Sciences (Ed Remington JP and Gennaro AR; Mack Pub. Co. Easton, Pa 1990).

Most preferably, however, the pharmaceutically acceptable excipient or carrier comprises a medium-chain triglyceride (MCT), preferably MCT oil. Suitable MCTs may be selected from a group consisting of a triglycerol linker with three fatty acid residues with carbon chain lengths ranging from 1 to 14. The chemical formula of each of these MCTs is shown below:

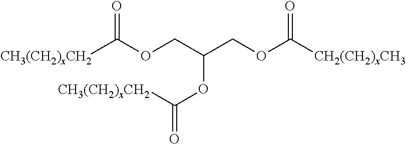

where X=1-14.

The MCT oil is preferably sourced from fractionated coconut oil. Preferred triglycerides comprise a carbon chain length or C6, C8, C10 or C12. Preferably, the triglyceride is almost all C8, which is highly refined.

The concentration of MCT oil in the cannabinoid-based formulation may be at least 80% (w/v), at least 82% (w/v) or at least 84% (w/v). Preferably, the concentration of MCT oil in the cannabinoid-based formulation may be at least 86% (w/v), or at least 88% (w/v). The concentration of MCT oil in the cannabinoid-based formulation may be less than 96% (w/v), less than 95% (w/v) or less than 94% (w/v). The concentration of MCT oil in the cannabinoid-based formulation may be less than 92% (w/v), or less than 90% (w/v). The concentration of MCT oil in the cannabinoid-based formulation may be between 85 and 93% (w/v), between 86 and 92% (w/v) or between 87 and 91% (w/v). Preferably, the concentration of MCT oil in the cannabinoid-based formulation is about 88.50% (w/v). It will be appreciated that any of the lower values for the concentration of MCT oil mentioned herein may be combined with any of the upper values for the concentration of MCT oil.

The cannabinoid-based formulation of the invention is preferably in a liquid form. The formulation may comprise a tincture.

Preferably, the formulation is administrable by sublabial, buccal, sublingual, or oropharyngeal administration delivery. The preparation of the liquid formulations for sublabial, buccal, oropharyngeal or sublingual (oral) delivery allows the introduction of the active agents (i.e. the cannabinoid and the terpenes) into the systemic circulation via a mucous membrane, increasing the onset of activity and potency due to bypassing first-pass metabolism and increasing the drug bioavailability. Advantageously, given the lipophilicity and poor bioavailability of oral administration of cannabinoids for gastrointestinal absorption, which is considered the most popular method currently in the market for the delivery of medicines, the formulations of the invention surprisingly enable administration by this route. The liquid formulations may also be prepared for inhaled or nebulised delivery to the respiratory tract, as well as formulations for topical application to the skin and mucosal surfaces.

The formulation may be administered at least once a day, at least once a week, or at least once a month. The formulation may be administered two or three times a day, two or three times a week, or two or three times a month.

The formulation may be administered to a “subject” in need of the treatment, which may be a vertebrate, mammal, or domestic animal. Hence, the formulations according to the invention may be used to treat any mammal, for example livestock (e.g. a horse), domestic pets, or may be used in other veterinary applications. Most preferably, however, the subject is a human being.

A “therapeutically effective amount” of the formulation is any amount which, when administered to the subject, is the amount of the aforementioned that is needed to treat the target medical condition.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:

FIG. 1 is a flow diagram showing a first embodiment of a method for producing a CBD formulation of the invention comprising a CBD isolate, terpene isolates and an MCT oil, as excipient.

FIG. 2 is a flow diagram showing a second embodiment of a method for producing a CBD formulation of the invention comprising a CBD isolate, terpene isolates and an MCT oil, as excipient.

FIG. 3 shows a Certificate of Analysis (COA) for one embodiment of the CBD raw material.

### EXAMPLES

### Background

The inventors have devised a novel method for reproducibly and consistently producing a highly standardised formulation comprising a cannabinoid (e.g. CBD) and a mixture of specific terpenes from scratch. The method involves combining an isolate of CBD of known purity, a highly defined mix of terpenes of known purity, and a medium chain triglycerides (MCT) oil of known purity, allowing the synthesis of a formulation with precisely standardised amounts of each component, whilst simultaneously minimising any contamination with THC.

### Example 1—Method of Producing the CBD Formulation

Referring to FIGS. 1 and 2, the inventors have designed a novel method for producing a highly uniform and reproducible CBD-based formulation. The method involves the following steps:
1. Extracting CBD from a Cannabis sativa plant to produce a CBD extract;
2. Purifying a CBD isolate (at a defined purity) from the CBD extract;
3. Extracting terpenes from the appropriate species of plant to produce a terpene extract (i.e. an essential oil);
4. Purifying a terpene (at a defined purity) from the terpene extract, several of which are then blended together to create a terpene blend;
5. Preparing an MCT oil (at a defined purity);
6. Mixing together the CBD isolate, the terpene blend and the MCT oil to produce the CBD formulation of the invention.

Each of these steps will now be described in detail.

### Example 2—Preparing the CBD Isolate

The inventors have used two different embodiments of a method for extracting the pure CBD isolate, i.e. solvent extraction (shown in FIG. 1) and enzyme extraction (shown in FIG. 2).

### Solvent Extraction (FIG. 1)

Referring to FIG. 1 (left-hand side), in a first embodiment, the CBD isolate is prepared with the use of solvent extraction (e.g. using either an alcohol or an alkane) from suitable plant material (Cannabis sativa inflorescence and/or leaves), producing a slurry which is then filtered to produce a crude liquid extract. The crude extract is chilled to preferably−80° C. for 24 hours in a process called “Winterisation” in order to precipitate fats and waxes that are subsequently filtered off to produce a so-called ‘winterised’ extract.

The solvent (i.e. alcohol or alkane) is then removed under reduced pressure and the remaining crude oil ‘decarboxylated’ to convert the acidic compounds to neutral forms, and this is typically performed at 120° C. for at least 30 minutes, ideally at least one hour. The resulting decarboxylated extract is further purified by short path distillation at 130° C.-180° C. to remove residual extraneous non-cannabinoid materials. The resulting material is dissolved in alcohol (i.e. a solvent) to around the saturation point of the solution, before an alkane (acting as an anti-solvent) is slowly added to induce crystallisation. Upon completion of the first crystallisation (~95% purity CBD), the solid material is filtered off and re-dissolved in alcohol, and then a second crystallisation is performed to produce isolated CBD of the required specification.

### Enzyme Extraction (FIG. 2)

With reference to FIG. 2, in a second embodiment, the one or more cannabinoid is prepared by using enzyme digestion of plant material (e.g. inflorescence and/or leaves of Cannabis sativa). The plant material can comprise a moisture content of about 40%. The plant material is contacted with an enzyme solution which comprises a combination of one or more of the following enzymes: cellulase, beta-glucosidase, hemicellulase, xylanase, glucanase, beta-glucanase, pectinase, amylase, alpha-amylase, phospholipase, beta-mannanase, arabinanase, phytase and protease. The pH of the enzyme solution is adjusted to about pH5.6 using citric acid. A weight ratio of 1-10% (wt) of plant material is used to 99-10% (wt) enzyme solution. The plant material is macerated before, during and/or after contacting with the enzyme solution. In addition to the enzyme solution, the plant material is also contacted with a lipid for extracting the cannabinoid (i.e. CBD). For example, the lipid is vegetable oil, MCT oil, seed oil or olive oil. The weight ratio of lipid to plant material is about 1:1.5. The plant material is also macerated before, during and/or after contacting with the lipid.

The mixture of plant material, enzymes and lipid is then stirred and macerated at 10° C.-50° C. for about an hour. Upon full breakdown and dissolution of the plant material, the oil and water phases are then separated by gravity, centrifugation, filtration or a combination thereof. The oil layer is transferred to a separate vessel having agitation capability. The oil layer is contacted with an aqueous base, such as 0.1M NaOH or KOH, at a pH greater than or equal to 12. The weight of aqueous basic solution relative to the cannabinoid-containing oil is 1:5. This mixture is agitated for at least 20 minutes at a temperature of 40° C.-80° C.

On completion of the previous step, the mixture is contacted with a combination of: (i) sodium chloride and/or calcium carbonate; and (ii) glucose and/or fructose, and then agitated for a further 20 minutes. Upon completion, the layers are separated by gravity or centrifugation, and the aqueous layer may then be separated off. The pH of the separated aqueous layer is reduced to a pH of about 1 by contacting the aqueous layer with 85% ortho-phosphoric acid. The solution is agitated for a further 20 minutes, causing precipitation of cannabinoid acid salts. This liquid is filtered, giving a cannabinoid-rich solid precipitate between 65%-90% total cannabinoid content. The cannabinoid-rich precipitate is heated to at least 120° C. for 60 mins, or until such time that complete decarboxylation has been achieved. The resulting material is dissolved in a solvent (e.g. alcohol) to around the saturation point of the solution, before an anti-solvent (e.g. alkane) is (slowly) added to induce crystallisation. Optionally, the solid material can be filtered off and re-dissolved in solvent (e.g. alcohol), and a second crystallisation may be performed to produce an isolated cannabinoid (preferably, CBD) of the required specification.

### Certificate of Analysis (COA)

Referring to FIG. 3, there is shown a Certificate of Analysis (COA) for one embodiment of the CBD raw material. As can be seen, a wide number of parameters have been measured, and are well within accepted limits. In particular, the inventors note the ability to set the specification for d9-tetrahydrocannabinol (THC) to 0.02%, which is significantly lower than the pharmaceutical requirements of less than, or equal to, 0.15% for any single known organic impurity.

### Example 3—Preparing the Terpene Blend

Referring to FIGS. 1 and 2 (right-hand side), the terpenes are prepared by_steam distillation to produce an essential oil from the appropriate species of plant, such as lavender. The resulting essential oil is purified to individual components by flash chromatography with silica. The following example is for the purification of linalool from lavender, though it will be appreciated that other essential oils comprising one or more terpene can be prepared from other plant species.

The steam distillation equipment is a closed system consisting of a boiling vessel into which macerated lavender is placed, adequate water is then added to allow for mobilisation of the plant material. Steam is introduced into the bottom of the vessel via a dip tube running from the top of the boiling vessel. The mixture is heated to boiling point and the vapour travels through a manifold at the top of the boiling vessel to a water-cooled condenser. The condensate is collected and allowed to separate, where the top essential oil layer is collected.

The essential oil is dissolved at the appropriate concentration in 90:10 heptane: ethyl acetate and introduced to a flash chromatography system and separated using a silica packed column and 90:10 heptane: ethyl acetate mobile phase. The later fractions yield >95% purity linalool.

The inventors used isolated terpenes after the purification process as raw materials for preparation of the CBD formulation. The purity standard of the raw terpenes is 95% purity and above (measured by GC). Impurities are mostly other terpenes, and all impurities above 0.2% are identified in order to be included as part of the formulation and to ensure the highest consistency.

The terpenes are then combined by weight to produce a highly reproducible terpene mixture or blend. As shown in FIGS. 1 and 2, the resulting essential oils, each comprising a terpene at a 95% purity or above, are mixed together to form a terpene blend, i.e. terpene X, terpene Y and/or terpene Z, and so on), each at a purity of 95% or more. Table 2 shows one exemplary embodiment of the terpene blend.

TABLE 2
Terpene blend

| Ingredient | % |
|---|---|
| Alpha-Pinene | 6.70 |
| Beta-Pinene | 6.70 |
| Limonene | 6.70 |
| Cis-Ocimene | 6.70 |
| Beta-Caryophyllene | 6.70 |
| Alpha-Humulene | 6.70 |
| Beta-Myrcene | 6.70 |
| Terpinolene | 6.70 |
| Trans beta Farnesene | 6.60 |
| Linalool | 6.70 |
| Bisabolol | 6.60 |
| Alpha-Phellandrene | 6.60 |
| Gamma-Terpinene | 6.60 |
| Alpha-Terpineol | 6.60 |
| Geraniol | 6.70 |
| Total | 100.00 |

### Example 4—Preparing the MCT Oil

Referring to FIGS. 1 and 2 (left-hand side), a commercially available pharmaceutical grade MCT oil is obtained from H Plus Limited, Walker House, Exchange Flags, Liverpool, L2 3YL, England. For example, the MCT can be sourced from fractionated coconut oil, and has a carbon chain length of C6, C8, C10 or C12, though a highly refined C8 chain length is preferred.

### Example 5—CBD Formulation

The method comprises combining the CBD isolate from Example 2, the terpene isolate from Example 3, and the MCT oil from Example 4 to produce the cannabinoid-based formulation of the invention. This is achieved by first mixing the CBD isolate with the MCT oil with agitation at about 20° C.-50° C. Then, the CBD/MCT solution is mixed with the plurality of blended terpenes with agitation at about 20° C.-50° C. to create the CBD/MCT/terpene formulation.

The inventors have designed an embodiment of the CBD-based formulation with the composition shown in Table 3.

TABLE 3
CBD Formulation

| | % Content | % Low | % High |
|---|---|---|---|
| Cannabinoid | | | |
| CBD | 10 | 8 | 12 |
| Terpenes | | | |
| Alpha-pinene | 0.1 | 0.01 | 0.2 |
| Beta-pinene | 0.1 | 0.01 | 0.2 |
| limonene | 0.1 | 0.01 | 0.2 |
| Cis-ocimene | 0.1 | 0.01 | 0.2 |
| Beta-caryophyllene | 0.1 | 0.01 | 0.2 |
| Alpha-humulene | 0.1 | 0.01 | 0.2 |
| Beta-myrcene | 0.1 | 0.01 | 0.2 |
| terpinolene | 0.1 | 0.01 | 0.2 |
| Trans beta farnesene | 0.1 | 0.01 | 0.2 |
| Linalool | 0.1 | 0.01 | 0.2 |
| Bisabolol | 0.1 | 0.01 | 0.2 |
| Alpha-Phellandrene | 0.1 | 0.01 | 0.2 |
| Gamma-Terpinene | 0.1 | 0.01 | 0.2 |
| Alpha-Terpineol | 0.1 | 0.01 | 0.2 |
| Geraniol | 0.1 | 0.01 | 0.2 |
| MCT Oil | 88.50 | | |
| Total | 100 | | |

### CONCLUSIONS

The resultant CBD formulations are referred to as Cannabis-Based Products for Medicinal use (CBPM) in humans or animals. In particular, the CBPMs are formulations containing cannabidiol (CBD) isolate, terpenes and medium-chain triglyceride (MCT) oil in a liquid form for sublabial, buccal, sublingual, or oropharyngeal administration delivery. The use of these formulations focuses primarily on conditions associated with pain and sleep disorders.

The formulations (or CBPMs) are broad-spectrum CBD mixtures, which consist of a particular combination of CBD and terpenes, produced at pharmaceutical standards under the guidelines of Good Manufacturing Practice (GMP) and designed to produce precise physical effects. To date, there are no products available in the UK that comply with the GMP standard, and so the pharmaceutical formulations of the invention address this gap and facilitate the access and delivery of a pharmaceutical standard CBPM for use in clinical trials and for treating patients.

The preparation of these liquid formulations for sublabial, buccal or sublingual delivery allows the introduction of the drug into the systemic circulation via a mucous membrane, increasing the onset of activity and potency due to bypassing first-pass metabolism and increasing the drug bioavailability. Advantageously, given the lipophilicity and poor bioavailability of oral administration of cannabinoids for gastrointestinal absorption, which is considered the most popular method currently in the market for the delivery of medicines, the formulations of the invention surprisingly enable administration by this route.

The active pharmaceutical components of the CBPM formulations are the CBD and terpenes. The MCT oil is acting as a pharmaceutically acceptable carrier, excipient or diluent and is, therefore, not considered to be an active component. The active components are believed to interact with the endocannabinoid system (ECS) and modulate a plethora of physiological responses. The ECS is a complex lipid cell-signalling system comprised of the cannabinoid receptors (CBRs) 1 and 2, the endogenous cannabinoids (endocannabinoids), anandamide (N-arachidonoylethanolamide, AEA) and 2-Arachidonoylglycerol (2-AG), the anandamide transporter protein (TP), and the enzymes responsible for the synthesis and degradation of endocannabinoids (fatty acid amide hydrolase, FAAH; and monoacylglycerol lipase, MAGL). However, further research into the endocannabinoid system pharmacology has led to the discovery of a variety of molecular targets for cannabinoids and terpenes independent of the CBRs 1 and 2, such as the transient receptor potential (TRP) channels, two orphan G-protein coupled receptors (GPR55 and GPCR18) and the peroxisome proliferator-activated receptor (PPAR). The ECS regulates homeostasis in various physiological processes such as inflammation, pain, appetite, metabolism, and memory.

The ECS orchestrates a ‘retrograde negative feedback mechanism’ in the CNS. Upon neuronal depolarisation, AEA and 2-AG are synthesised on the postsynaptic terminal in dendritic spines and somatodendritic compartments. They are then released into the neuronal cleft to suppress the inhibitory neurotransmitter gamma-aminobutyric acid (GABA) secretion in GABAergic afferents and the excitatory neurotransmitter glutamate in glutamatergic neurons. Additionally, eCB signalling in non-neuronal tissues regulates several physiological processes, such as spermatogenesis, pain and the modulation of the immune system. Given that the ECS modulates multiple cell functions, which play critical roles in pain, neuromodulation, mood, immunity and other physiological processes, therapeutics targeting this system, such as the CBPMs described herein, hold potential as novel approaches in treating various conditions, such as chronic pain, anxiety and inflammation.

CBPMs have been added to the treatment paradigm for many patients suffering from acute and chronic inflammatory conditions, pain, and symptoms of negative affect such as anxiety and depression that have limited or a lack of response to traditional therapeutic approaches. Due to this, the National Institute for Health and Care Excellence (NICE) and the International Association for the Study of Pain (IASP) have made specific calls for research using CBPMs in humans.

Therefore, there is a current unmet need for treating multiple treatment-resistant conditions, such as fibromyalgia, chemotherapy-induced peripheral neuropathy (CIPN) and endometriosis which are characterised by pain, inflammation and negative affect. Since no one-size-fits-all therapy is available, most patients require multiple and alternative treatment approaches. Additionally, numerous CBD formulations are now available as food supplements, making many beneficial health claims, with minimal assessment of whether they are being administered in a suitable formulation even to achieve blood levels to support these claims. The method of the invention addresses this need and produces a medical quality CBPM at pharmaceutical standards and under the guidelines of GMP aimed for its use in medical research and practice.

1. A method of producing a cannabinoid-based formulation comprising a cannabinoid and substantially lacking tetrahydrocannabinol (THC), the method comprising combining: (i) one or more cannabinoid; and (ii) a plurality of terpenes, to produce a cannabinoid-based formulation, wherein the formulation substantially lacks THC.

2. The method according to claim 1, wherein the concentration of THC in the formulation is less than 0.01% (w/v), less than 0.005% (w/v), or less than 0.001% (w/v).

3. The method according to either claim 1 or claim 2, wherein the concentration of THC in the formulation is less 0.0005% (w/v), less than 0.0002% (w/v), or less than 0.0001% (w/v).

4. The method according to any preceding claim, wherein the formulation comprises no detectable THC using reasonable means of analysis.

5. The method according to any preceding claim, wherein the one, two or three cannabinoids selected from a group of cannabinoids consisting of: cannabidiol (CBD); cannabichromene (CBC); cannabigerol (CBG); and cannabigerol monomethyl ether (CBGM).

6. The method according to any preceding claim, wherein the one or more cannabinoid comprises CBD.

7. The method according to any preceding claim, wherein the one or more cannabinoid comprises an isolate of cannabidiol (CBD).

8. The method according to any preceding claim, wherein the formulation does not comprise CBC, CBG, or CBGM.

9. The method according to any preceding claim, wherein the one or more cannabinoid is prepared using solvent extraction or enzyme digestion of plant material, preferably inflorescence and/or leaves of Cannabis sativa.

10. The method according to claim 9, wherein the plant material is contacted with an enzyme solution comprising one or more enzyme selected from a group of enzymes consisting of: cellulase, beta-glucosidase, hemicellulase, xylanase, glucanase, beta-glucanase, pectinase, amylase, alpha-amylase, phospholipase, beta-mannanase, arabinanase, phytase and protease, optionally wherein the pH of the enzyme solution is adjusted to at least about pH4.

11. The method according to either claim 9 or claim 10, wherein the plant material is contacted with a lipid, optionally vegetable oil, MCT oil, seed oil or olive oil, then agitated for at least 30, 40 or 60 minutes at a temperature of between about 5° C. and 80° C., between 8° C. and 65° C., or between 10° C. and 50° C., and then the oil and water phases are separated by gravity, centrifugation, filtration or a combination thereof.

12. The method according to claim 11, wherein the oil layer is contacted with an aqueous base, agitated for at least 10-20 minutes at a temperature of between about 20° C. and 100° C., between 30° C. and 90° C., or between 40° C. and 80° C., and then contacted with a combination of: (i) sodium chloride and/or calcium carbonate; and (ii) glucose and/or fructose, optionally wherein this mixture is agitated for a further 10, 15 or 20 minutes.

13. The method according to claim 12, wherein the layers are separated by either gravity or centrifugation, and the aqueous layer is then separated off, and the pH of the separated aqueous layer is reduced to a pH of less than 3, 2 or 1.

14. The method according to claim 13, wherein the solution is agitated for a further 10, 15 or 20 minutes, causing precipitation of cannabinoid acid salts, wherein this liquid is filtered, giving a cannabinoid-rich solid precipitate, optionally wherein cannabinoid-rich precipitate is heated to at least 90, 100 or 120° C. for at least 30 mins, 45 mins or 60 mins, or until such time that complete decarboxylation has been achieved.

15. The method according to claim 14, wherein the resulting material is dissolved in a solvent to around the saturation point of the solution, before being contacted with an anti-solvent to induce crystallisation, optionally wherein the solid material is filtered off and re-dissolved in solvent, and a second crystallisation is performed to produce an isolated cannabinoid (preferably, CBD) of the required specification.

16. The method according to any preceding claim, wherein the purity of the one or more cannabinoid is at least 97%, at least 98% or at least 99%, or about 98-100%.

17. The method according to any preceding claim, wherein the concentration of the one or more cannabinoid, preferably CBD, in the cannabinoid-based formulation is at least 2% (w/v), at least 3% (w/v) or at least 4% (w/v).

18. The method according to any preceding claim, wherein the concentration of the one or more cannabinoid, preferably CBD, in the cannabinoid-based formulation is at least 5% (w/v), at least 6% (w/v) or at least 7% (w/v).

19. The method according to any preceding claim, wherein the concentration of the one or more cannabinoid, preferably CBD, in the cannabinoid-based formulation is at least 8% (w/v), at least 9% (w/v) or at least 10% (w/v).

20. The method according to any preceding claim, wherein the concentration of the one or more cannabinoid, preferably CBD, in the cannabinoid-based formulation between 7 and 13% (w/v), or between 8 and 12% (w/v).

21. The method according to any preceding claim, wherein the concentration of the one or more cannabinoid, preferably CBD, in the cannabinoid-based formulation between 9 and 11% (w/v).

22. The method according to any preceding claim, wherein the plurality of terpenes is prepared by first steam distillation from the appropriate species of plant to produce an essential oil, and then the resulting essential oil is then purified by chromatography, preferably flash chromatography, to separate the individual terpene components at the required purity.

23. The method according to any preceding claim, wherein the purity of the plurality of terpenes is at least 90%, at least 92% or at least 95% of total terpenes.

24. The method according to any preceding claim, wherein the plurality of terpenes is selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.

25. The method according to any preceding claim, wherein the plurality of terpenes comprises:
(i) at least three terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene;
(ii) at least four terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene;
(iii) at least five terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene;
(iv) at least six terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene;
(v) at least seven terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.
(vi) at least eight terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene;
(vii) at least nine terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene;
(viii) at least ten terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.
(ix) at least eleven terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene;
(x) at least twelve terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene;
(xi) at least thirteen terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene;
(xii) at least fourteen terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene; and/or
(xiii) at least fifteen terpenes selected from a group of terpenes consisting of: Alpha-Pinene, Beta-Pinene, Limonene, Cis-Ocimene, Beta-Myrcene, Terpinolene, Linalool, Alpha-Phellandrene, Gamma-Terpinene, Alpha-Terpineol, Borneol, Camphene, Eucalyptol, Fenchol, Beta-Caryophyllene, Alpha-Humulene, Trans Beta Farnasene, Bisabolol, Caryophyllene Oxide, Nerolidol, Guaiol, Cedrol, Beta-Eudesmol, Santalene, Bergomotene, Alpha-Guaiene, Geraniol and Bisbolene.

26. The method according to any preceding claim, wherein the plurality of terpenes comprises at least two, three, four or five terpenes selected from a group of terpenes consisting of: Alpha-Pinene, Beta-Pinene, Limonene, Cis-Ocimene, Beta-Caryophyllene, Alpha-Humulene, Beta-Myrcene, Terpinolene, Trans Beta Farnasene, Linalool, Bisabolol, Alpha-Phellandrene, Gamma-Terpinene, Geraniol and Alpha-Terpineol.

27. The method according to any preceding claim, wherein the plurality of terpenes comprises at least six, seven or eight terpenes selected from a group of terpenes consisting of: Alpha-Pinene, Beta-Pinene, Limonene, Cis-Ocimene, Beta-Caryophyllene, Alpha-Humulene, Beta-Myrcene, Terpinolene, Trans Beta Farnasene, Linalool, Bisabolol, Alpha-Phellandrene, Gamma-Terpinene, Geraniol and Alpha-Terpineol.

28. The method according to any preceding claim, wherein the plurality of terpenes comprises at least nine, ten or eleven terpenes selected from a group of terpenes consisting of: Alpha-Pinene, Beta-Pinene, Limonene, Cis-Ocimene, Beta-Caryophyllene, Alpha-Humulene, Beta-Myrcene, Terpinolene, Trans Beta Farnasene, Linalool, Bisabolol, Alpha-Phellandrene, Gamma-Terpinene, Geraniol and Alpha-Terpineol.

29. The method according to any preceding claim, wherein the plurality of terpenes comprises at least twelve, thirteen, fourteen or fifteen terpenes selected from a group of terpenes consisting of: Alpha-Pinene, Beta-Pinene, Limonene, Cis-Ocimene, Beta-Caryophyllene, Alpha-Humulene, Beta-Myrcene, Terpinolene, Trans Beta Farnasene, Linalool, Bisabolol, Alpha-Phellandrene, Gamma-Terpinene, Geraniol and Alpha-Terpineol.

30. The method according to any one of claims 24-29, wherein:
(i) the concentration of α-Pinene in the cannabinoid-based formulation is at least 0.0001 (w/v), at least 0.001% (w/v), less than 0.75% (w/v), less than 0.5% (w/v), less than 0.3% (w/v), between 0.0001 and 0.5% (w/v), between 0.01 and 0.3% (w/v) or between 0.05 and 0.2% (w/v);
(ii) the concentration of β-Pinene in the cannabinoid-based formulation is at least 0.0001 (w/v), at least 0.001% (w/v), less than 0.75% (w/v), less than 0.5% (w/v), less than 0.3% (w/v), between 0.0001 and 0.5% (w/v), between 0.01 and 0.3% (w/v) or between 0.05 and 0.2% (w/v);
(iii) the concentration of Limonene in the cannabinoid-based formulation is at least 0.0001 (w/v), at least 0.001% (w/v), less than 0.75% (w/v), less than 0.5% (w/v), less than 0.3% (w/v), between 0.0001 and 0.5% (w/v), between 0.01 and 0.3% (w/v) or between 0.05 and 0.2% (w/v);
(iv) the concentration of Cis-Ocimene in the cannabinoid-based formulation is at least 0.0001 (w/v), at least 0.001% (w/v), less than 0.75% (w/v), less than 0.5% (w/v), less than 0.3% (w/v), between 0.0001 and 0.5% (w/v), between 0.01 and 0.3% (w/v) or between 0.05 and 0.2% (w/v);
(v) the concentration of β-Caryophyllene in the cannabinoid-based formulation is at least 0.0001 (w/v), at least 0.001% (w/v), less than 0.75% (w/v), less than 0.5% (w/v), less than 0.3% (w/v), between 0.0001 and 0.5% (w/v), between 0.01 and 0.3% (w/v) or between 0.05 and 0.2% (w/V);
(vi) the concentration of Alpha-Humulene in the cannabinoid-based formulation is at least 0.0001 (w/v), at least 0.001% (w/v), less than 0.75% (w/v), less than 0.5% (w/v), less than 0.3% (w/v), between 0.0001 and 0.5% (w/v), between 0.01 and 0.3% (w/v) or between 0.05 and 0.2% (w/V);
(vii) the concentration of Myrcene in the cannabinoid-based formulation is at least 0.0001 (w/v), at least 0.001% (w/v), less than 0.75% (w/v), less than 0.5% (w/v), less than 0.3% (w/v), between 0.0001 and 0.5% (w/v), between 0.01 and 0.3% (w/v) or between 0.05 and 0.2% (w/v);
(viii) the concentration of Terpinolene in the cannabinoid-based formulation is at least 0.0001 (w/v), at least 0.001% (w/v), less than 0.75% (w/v), less than 0.5% (w/v), less than 0.3% (w/v), between 0.0001 and 0.5% (w/v), between 0.01 and 0.3% (w/v) or between 0.05 and 0.2% (w/v);
(ix) the concentration of Farnesene in the cannabinoid-based formulation is at least 0.0001 (w/v), at least 0.001% (w/v), less than 0.75% (w/v), less than 0.5% (w/v), less than 0.3% (w/v), between 0.0001 and 0.5% (w/v), between 0.01 and 0.3% (w/v) or between 0.05 and 0.2% (w/V);
(x) the concentration of Linalool in the cannabinoid-based formulation is at least 0.0001 (w/v), at least 0.001% (w/v), less than 0.75% (w/v), less than 0.5% (w/v), less than 0.3% (w/v), between 0.0001 and 0.5% (w/v), between 0.01 and 0.3% (w/v) or between 0.05 and 0.2% (w/v);
(xi) the concentration of Bisabolol in the cannabinoid-based formulation is at least 0.0001 (w/v), at least 0.001% (w/v), less than 0.75% (w/v), less than 0.5% (w/v), less than 0.3% (w/v), between 0.0001 and 0.5% (w/v), between 0.01 and 0.3% (w/v) or between 0.05 and 0.2% (w/v);
(xii) the concentration of Alpha-Phellandrene in the cannabinoid-based formulation is at least 0.0001 (w/v), at least 0.001% (w/v), less than 0.75% (w/v), less than 0.5% (w/v), less than 0.3% (w/v), between 0.0001 and 0.5% (w/v), between 0.01 and 0.3% (w/v) or between 0.05 and 0.2% (w/v);
(xiii) the concentration of Gamma-Terpinene in the cannabinoid-based formulation may be at least 0.0001 (w/v), at least 0.001% (w/v), less than 0.75% (w/v), less than 0.5% (w/v), less than 0.3% (w/v), between 0.0001 and 0.5% (w/v), between 0.01 and 0.3% (w/v) or between 0.05 and 0.2% (w/V);
(xiv) the concentration of Geraniol in the cannabinoid-based formulation is at least 0.0001 (w/v), at least 0.001% (w/v), less than 0.75% (w/v), less than 0.5% (w/v), less than 0.3% (w/v), between 0.0001 and 0.5% (w/v), between 0.01 and 0.3% (w/v) or between 0.05 and 0.2% (w/v); and/or
(xv) the concentration of Alpha-Terpineol in the cannabinoid-based formulation is at least 0.0001 (w/v), at least 0.001% (w/v), less than 0.75% (w/v), less than 0.5% (w/v), less than 0.3% (w/v), between 0.0001 and 0.5% (w/v), between 0.01 and 0.3% (w/v) or between 0.05 and 0.2% (w/v).

31. The method according to any preceding claim, wherein the method comprises combining the cannabinoid and plurality of terpenes with a pharmaceutically acceptable excipient or carrier.

32. The method according to claim 31, wherein the pharmaceutically acceptable excipient or carrier comprises a medium-chain triglyceride (MCT), preferably MCT oil.

33. The method according to claim 32, the chemical formula of the MCT is shown below: 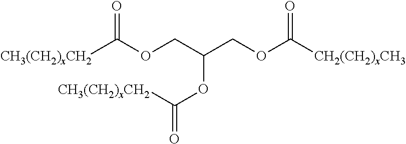
wherein X=1-14.

34. The method according to any one of claims 31-33, wherein the method comprises contacting the cannabinoid with the pharmaceutically acceptable excipient or carrier at a temperature of between about 10° C. and 70° C., between about 15° C. and 60° C., or between about 20° C. and 50° C., to form a mixture of the cannabinoid and excipient or carrier.

35. The method according to claim 34, wherein the mixture of cannabinoid and excipient or carrier is contacted with the plurality of terpenes at a temperature of between about 10° C. and 70° C., between about 15° C. and 60° C., or between about 20° C. and 50° C., to create the cannabinoid-based formulation.

36. The method according to any one of claims 32-35, wherein the concentration of MCT oil in the cannabinoid-based formulation is at least 80% (w/v), at least 82% (w/v), at least 84% (w/v), less than 96% (w/v), less than 95% (w/v), less than 94% (w/v), between 85 and 93% (w/v), between 86 and 92% (w/v) or between 87 and 91% (w/v).

37. The method according to any preceding claim, wherein the cannabinoid-based formulation is in a liquid form, and optionally comprises a tincture.

38. The method according to any preceding claim, wherein the formulation is administrable by sublabial, buccal, sublingual, or oropharyngeal administration delivery.

39. A cannabinoid-based formulation obtained or obtainable by the method of any one of claims 1-38.

40. The cannabinoid-based formulation according to claim 39, for use in therapy.
